(19) United States
(12) Patent Application Publication
(10) Pub. No.: US 2010/0185102 A1
(43) Pub. Date: Jul. 22, 2010

(54) MOVEMENT DETECTION DURING RADIATION TREATMENT USING A RADAR SYSTEM

(76) Inventor: Matthias SAAR, Altendorf (DE)

Correspondence Address: BRINKS HOFER GILSON & LIONE, P.O. BOX 10395, CHICAGO, IL, 60610, US

(21) Appl. No.: 12/686,084

(22) Filed: Jan. 12, 2010

(30) Foreign Application Priority Data
Jan. 19, 2009 (DE) DE 102009005110.4

(51) Int. Cl. A61B 5/0205; A61N 5/10; G01S 13/00

(52) U.S. Cl. 600/484; 250/492.1; 250/492.3; 342/22; 342/21

(57) ABSTRACT

The present embodiments relate to irradiating a patient with an irradiation source directed onto the patient, as well as with a radar system for detecting the position of the patient or a part of the patient during the irradiation.

The present patent document claims the benefit of the filing date of DE 10 2009 005 110.4 filed on Jan. 19, 2009, which is hereby incorporated by reference.

### BACKGROUND

The present embodiments relate to irradiating a patient with a radiation source directed onto the patient.

In particle therapy, which can be used for therapy of cancers, a tumor is irradiated precisely with a proton or an ion beam. In contrast to irradiation with electromagnetic beams, with ion irradiation the majority of the beam energy is discharged directly in the tumor. The tissue lying above the tumor incurs less damage. The chances of healing are no worse than with a therapy with other beams but far fewer side effects arise. Frequently carbon and neon ions are used.

In order to damage as little healthy tissue as possible, the patient is supported so that the particles interact with the desired region of the body, for example, with the tumor tissue, and as little as possible with the healthy tissue. A change in the position of the patient leads to this condition no longer being fulfilled. The same also applies to breathing and heart activity of the patient if the region to be irradiated lies in the chest or stomach area. This leads to an undesired irradiation of healthy tissue.

### SUMMARY AND DESCRIPTION

The present embodiments may obviate one or more of the drawbacks or limitations inherent in the related art. For example, in one embodiment, when irradiating a patient, the described negative effect of irradiating healthy tissue can be avoided. An apparatus, computer program, and a method for irridating a patient are presented.

In one embodiment, an apparatus for irradiating a patient includes a radiation source directed onto the patient and one or more radar systems for detecting the position of the patient or a part of the patient during the irradiation.

Electromagnetic waves are transmitted by a radar system or device as a primary signal and the “echoes” reflected from objects are received. By evaluating the received echoes, information about the reflecting object can be obtained.

The use of the radar system or of the radar systems serves to detect the position of a patient. This can involve an absolute or a relative position, for example, relative to the source of the radiation or the at least one radar system. The position or location of the patient as a whole can be of interest or also just a part of the patient, such as an organ or a tumor, for example. The position detection can be used in conjunction with the irradiation, since with an irradiation it is necessary to know which part of the patient the beam is hitting.

The position is detected during irradiation of the patient. The radar system or radar systems are designed and/or oriented such that the radar system or radar systems do not suffer any damage from the radiation. Accordingly, the irradiation does not have to be interrupted in order to undertake the position detection using the radar system or the radar systems. The irradiation can be adapted in an uninterrupted manner to a change in the position of the patient in the irradiated body region of the patient.

The apparatus can include one or more components, as described in greater detail in the embodiments and developments below.

The at least one radar system can be an ultra wide band (UWB) radar system. UWB radar systems have a good (sufficient) local resolution. The frequencies emitted by such a radar device do not present any danger to the human body.

In one embodiment, the apparatus can include an evaluation unit for determining the position of the patient or a part of the patient from data detected by the at least one radar system. The evaluation unit can employ suitable algorithms in order to calculate the desired position from the information provided by the at least one radar system. The evaluation unit can be used to establish changes in this position.

In one embodiment, the irradiation source is a particle-emitting source. The particles may be ions, such as carbon ions, for example, or protons.

One, two, or more radar systems can be used. The radar systems can emit their primary radiation onto the patient at different angles, for example, not in parallel to each other. This increases the local resolution.

In one embodiment, a control device is present for adjusting and/or interrupting the beam emitted by the radiation source as a result of the detected position of the patient or the part of the patient. The control device may be connected to the evaluation unit and controls the beam as a function of results of the evaluation unit. The adjustment of the beam corresponds to a change in its direction, whereas its interruption represents an at least temporary switching off of the beam. Such measures are especially advantageous if it is established that the position of the patient or of organs of the patient have changed. This enables the irradiation of a patient in undesired regions to be avoided. By suitably adjusting the beam, despite changes in a position of the patient or the part of the patient, the same section of the body can be irradiated.

In addition or as an alternative to the above evaluation unit for determining the position of the patient or the part of the patient, an evaluation unit can be present for determining from the data detected by the at least one radar system the position of a person other than the patient and/or a device. The other person in this case is likely to be one of the medical personnel monitoring the irradiation or the patient during the irradiation. Whether people or devices are located at dangerous or undesired positions within the irradiation room or are moving into such positions can be checked using the evaluation unit. It can be checked or established using the evaluation unit, whether there are other persons located within the irradiation room. This is because no-one other than the patient should be present in the treatment room during the irradiation.

A control device for adjusting and/or interrupting the beam emitted by the irradiation source as a result of the detected position of the person and/or the device can be provided. An interruption may occur, for example, if the person or the device is located in the vicinity of the beam or of the beam outlet opening. There can be sensitive electronic measuring devices located here which should not be touched. In addition or as an alternative to influencing the beam by interrupting and/or adjusting it, it is possible, as a result of the detected position of the person and/or the device, for the movement of the device to be changed or stopped, or for electronic units of the device to be switched off.

In addition or as an alternative to two evaluation units, an evaluation unit can be present for determining breathing and/or heart activity of the patient from data detected by the at least one radar system. This makes it possible to dispense with all or some of the devices for observing breathing and pulse of the patient during irradiation.

A control device for adjusting and/or interrupting the beam emitted by the radiation source as a result of the breathing and/or heart activity determined is of advantage. An interruption is sensible, for example, if a panic or disturbed state of the patient is detected based on the breathing and/or heart activity characteristic thereof.

The number of evaluation units described can be realized independently of each other. It is however also possible for a single evaluation unit be present which performs one, a number or all of the determination functions explained above.

In one embodiment, a computer program having the functionality of an input for data from at least one radar system for detecting the position of the patient or a part of the patient during the irradiation, as well as the functionality of an evaluation part for determining the position of the patient or the part of the patient from the data, is provided.

Also advantageous is the functionality of a control component for controlling the adjustment and/or interruption of the beam emitted by the irradiation source based on the position of the patient or the part of the patient determined.

Also possible is the functionality of an evaluation component for determining the position of a person and/or a device and/or for determining a breathing and/or heart activity of the patient from the data.

At least one radar system for detecting the position of a patient or a part of the patient during an irradiation is used in a detection method.

The information relating to the advantages, embodiment and developments of the apparatus correspondingly applies to the computer program and the method. To this end they can include further suitable functionalities or acts.

### BRIEF DESCRIPTION OF THE DRAWING

FIG. 1 illustrates one embodiment of a monitoring system.

### DETAILED DESCRIPTION

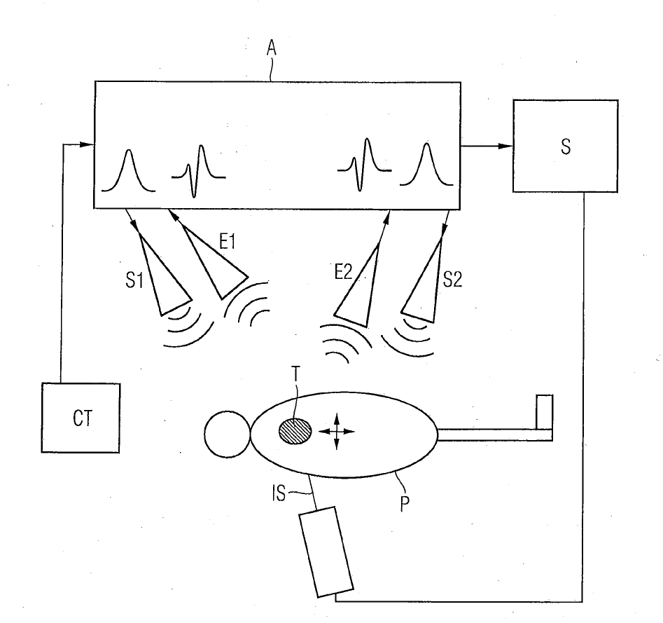

FIG. 1 shows a patient P who is being irradiated by an ion beam IS during particle therapy. The ion beam IS can be a beam of carbon ions, for example. A proton beam can also be employed. The objective of the irradiation is that the ion beam IS reaches the patient's tumor T and destroys the tumor T. The control device S serves to control the deflection of the ion beam IS in different directions. Using magnets, the direction of the ion beam IS can be changed by the control device S so that the ion beam IS can be directed precisely onto the tumor. Deflection of the ion beam IS is illustrated by the two double-ended arrows next to the tumor T.

During irradiation, the ion beam IS is aligned exactly on the tumor T and hits the target volume corresponding to the tumor T. A displacement of the ion beam IS by just a few millimeters would destroy parts of the healthy tissue and allows parts of the tumor T to survive, which would promote the continuance of the tumor T. Accordingly, it is necessary to know the position of the tumor T as precisely as possible for the irradiation.

The patient P is first fixed on a table plate in an immobilization room and is then transported by a shuttle system into the treatment room. A robot-based patient support apparatus (“table robot”) takes the table plate with the patient P and guides the table plate precisely into the desired position. Since the location of the tumor T within the patient P has been precisely determined beforehand by an imaging method such as computed tomography, for example, the position of the tumor T is known.

An x-ray system CT is present in the treatment room, for example, on the ceiling, for verification of the position of the tumor T. If the position detected by the x-ray system CT does not match the target position, the table robot makes an adjustment until the tumor T lies precisely in the isocenter.

This x-ray system CT is, however, not operated during the irradiation with the ion beam IS. Instead, the x-ray system CT is swung up onto the ceiling before the irradiation in order to be as far away as possible from the primary radiation. During the irradiation, there is no possibility of checking the position of the tumor T using computed tomography.

In the event that the tumor T is located on the head of the patient P, a patient mask can be used which fixes the head of the patient. The mask prevents the head of the patient from moving, so that the possibility of the ion beam IS missing the tumor T is almost excluded.

In the event that the tumor T, as shown in FIG. 1, is located in the stomach or chest area of the patient, despite external fixing of the patient P, the patient's stomach or chest still moves because of breathing and heart movements. These movements of the tumor T, where the ion beam IS is not adjusted for them, led to incorrect irradiation of the patient P.

Breathing and heart movements of the patient can be detected. Conclusions about the movement of the tumor T can be determined from these movements. Options for doing this are provided, for example, by laser systems which measure laser marks attached to the skin of the patient, or chest straps. The disadvantage is that only indirect information about the movements of the tumor T is produced from the methods. This is because the movement of the surface of the patient P generally does not coincide with that of the tumor T.

To check the position of the tumor T, which depends both on the possibly changeable position of the patient P and the breathing and heart activity, during the irradiation, two ultra wideband (UWB) radar systems are used. The first radar system includes a first transmitter S1 and a first receiver E1. The second radar system includes a second transmitter S2 and a second receiver E2. The first and second transmitters S1 and S2 and the first and second receivers E1 and E2 are connected to the evaluation unit A. The receiver E1 serves to detect the radiation emitted by the transmitter S1 and reflected by the patient P or by their surroundings. The same applies to the receiver E2 in relation to the radiation of the transmitter S2.

UWB systems use the properties of electromagnetic fields with an extremely large bandwidth, for example, approximately. 1-10 GHz in the 3-11 GHz frequency range. The transmit power is low, for example, less than 1 mW or less than 0.5 mW. UWB systems are used to obtain information about the state of their surroundings in a non-destructive, non-contact manner and with high resolution.

The first and second transmitters may be transmit antennas S1 and S2 that emit wideband electromagnetic pulses at low power and of short duration, for example, of less than a nanosecond. When these electromagnetic pulses hit the patient P, they penetrate into the human body and are partly reflected on consecutive boundary layers of different tissue types. Such boundary layers are produced, for example, by the transition from one organ to another organ. The reflected signals from different depths of the body are detected with the first and second receivers E1 and E2, which can be receive antennas. Since the different types of human tissue have typical absorption and reflection properties, the positions of the organs of the patient P can be detected precisely.

The position of the tumor T is checked by a computed tomography CT before the start of the irradiation. At this point in time, measurements should also be undertaken with the radar systems, so that with this a target measurement result of the radar measurements is known.

Movements of the patient P, as well as the patient's P breathing and heartbeat, displace and deform the boundary layers and thereby change the measured signal of the radar systems. The displacement of the organs of the patient P can be determined using suitable algorithms in the evaluation unit A. Measurement data is thus obtained from the anatomical movements from which the movements of organs can be reconstructed as a function of their location. By using the UWB radar systems, the movements of the organs deep within the body of the patient P may be determined in a non-contact manner. A chronological sequence of 3D images is produced. Since the position of the tumor T is known in relation to the organs, the current position of the tumor T can be determined from this relationship.

Just a single UWB system is in principle sufficient for this purpose. The use of two or more systems is however advantageous since in this manner redundant data will be obtained to enhance the safety and improve the local resolution. FIG. 1 shows the advantageous case in which the two transceiver systems are attached at an angle of 45 degrees and to the ceiling of the treatment room.

The evaluation unit A determines signals from the data of the receivers E1 and E2 which correspond to the movement of the tumor T. These signals can be used to control the ion beam IS using the control device S. Accordingly, the ion beam IS can be adjusted to the tumor so that the patient P is not irradiated incorrectly. The ion beam IS can follow the movement of the diseased organ and destroy the tumor T, without hitting healthy tissue. As an alternative to this it is possible to switch off the ion beam IS as soon as an organ or patient movement has been detected by the radar systems.

As well as the described application of observing the position of the tumor T, the radar systems can be used to other types of monitoring

For example, vital functions of the patient P, such as breathing and/or heartbeat, can be monitored non-invasively with the aid of the radar systems. This is done by observing the chest cavity using the radars. Other patient monitors, such as EKG devices, for example, are thus not needed in the treatment room during the irradiation. This is advantageous in as much as devices located in the treatment room can be hit by stray radiation during the irradiation of the patient P, so that there is the threat of radiation damage. The devices are then radioactive so that they must first be “decontaminated” before they are allowed to leave the treatment room again. Before such a device is removed from the treatment room it must be ensured that it is no longer radioactive.

A change in heartbeat and/or breathing detected by the radar systems can lead to the ion beam IS being interrupted. The ion beam IS can be interrupted, for example, if the patient panics, a state which becomes apparent through major changes in breathing and heart activity.

Furthermore there is also the possibility of preventing an unintentional approach by a person or a device to the ion beam IS or to sensitive electronic parts. The outlet opening of the ion beam IS can be monitored by the radar systems, for example. If anyone or anything approaches the outlet opening, the ion beam IS or an electronic unit is to be interrupted. In addition or as an alternative, an alarm can be triggered.

Finally the environment of movable components can also be monitored with a radar system. This includes robots or a range shifter, for example. If it is detected that a person or a device has moved into a zone in which there is a danger of a collision with the monitored movable component, the movement of this component can be stopped and/or an alarm triggered.

Basically the detection of movement of all devices and/or persons that are located within the detection range of the UWB radar systems is possible. This typically amounts to a few meters in diameter, for example, with a focus on the patient P, a few meters around the patient P to be irradiated.

The invention has been described above with reference to an exemplary embodiment. Naturally numerous changes and modifications a possible without departing from the framework of the invention.

1. An apparatus for irradiating a patient, the apparatus comprising:
a radiation source directed onto the patient, and
at least one radar system to detect a position of a patient or a part of the patient during the irradiation.

2. The apparatus as claimed in claim 1, wherein the at least one radar system includes a first radar system including a first transmitter and a first receiver.

3. The apparatus as claimed in claim 1, wherein the part of the patient is a tumor.

4. The apparatus as claimed in claim 1, wherein the at least one radar system is a ultra wide band (UWB) radar system.

5. The apparatus as claimed in claim 1, further comprising an evaluation unit for detecting the position of the patient of the part of the patient from data detected by the at least one radar system.

6. The apparatus as claimed in claim 1, wherein the radiation source is a particle-emitting source.

7. The apparatus as claimed in claim 6, wherein the particle-emitting source emits an ion beam.

8. The apparatus as claimed in claim 1, wherein the at least one radar system includes two radar systems.

9. The apparatus as claimed in claim 1, further comprising a control device for adjusting a beam emitted by the radiation source as a function of the detected position of the patient or the part of the patient.

10. The apparatus as claimed in claim 1, further comprising a control device for interrupting a beam emitted by the radiation source as a function of the detected position of the patient or the part of the patient.

11. The apparatus as claimed in claim 1, further comprising an evaluation unit for determining the position of the patient or a device from data detected by the at least one radar system.

12. The apparatus as claimed in claim 11, further comprising a control device for adjusting a beam emitted by the radiation source as a result of the position of the patient and/or the device determined.

13. The apparatus as claimed in claim 11, further comprising a control device for interrupting a beam emitted by the radiation source as a result of the position of the patient and/or the device determined.

14. The apparatus as claimed in claim 1, further comprising an evaluation unit for determining a breathing and/or heart activity of the patient from data detected by the at least one radar system.

15. The apparatus as claimed in claim 14, further comprising a control device for adjusting a beam emitted by the radiation source as a result of the breathing and/or heart activity determined.

16. The apparatus as claimed in claim 14, further comprising a control device for interrupting a beam emitted by the radiation source as a result of the breathing and/or heart activity determined.

17. A computer program comprising:
an evaluation component for determining a position of a patient or a part of the patient from the data with an input from at least one radar system to detect the position of the patient or the part of the patient during an irradiation.

18. The computer program as claimed in claim 17, further comprising a control component for controlling the adjustment and/or interruption of the beam emitted by the irradiation source on the basis of the position of the patient or the part of the patient determined.

19. The computer program as claimed in claim 17, further comprising a further evaluation component for determining the position of the patient and/or a device and for determining a breathing and/or heart activity of the patient from the data.

20. A method for position detection, the method comprising:
using at least one radar system for detecting a position of a patient or a part of the patient during a radiation process.
